(19) United States
(12) Patent Application Publication
(10) Pub. No.: US 2024/0398840 A1
(43) Pub. Date: Dec. 5, 2024

(54) SUCRALFATE-CONTAINING JELLY-LIKE PHARMACEUTICAL COMPOSITION

(71) Applicant: FUJI CHEMICAL INDUSTRIES CO., LTD., Nakaniikawa-gun (JP)

(72) Inventors: Haruka YONEDA, Nakaniikawa-gun (JP); Yo NAKASHIMA, Nakaniikawa-gun (JP); Yosuke HIRAI, Nakaniikawa-gun (JP); Yoshiro NAGAI, Nakaniikawa-gun (JP)

(73) Assignee: FUJI CHEMICAL INDUSTRIES CO., LTD., Nakaniikawa-gun (JP)

(21) Appl. No.: 18/691,357

(22) PCT Filed: Sep. 16, 2022
(86) PCT No.: PCT/JP2022/034694

§ 371 Date: Mar. 12, 2024

(30) Foreign Application Priority Data
Sep. 17, 2021 (JP) 2021-152273

(51) Int. Cl. A61K 31/7024; A61K 9/06; A61K 47/26; A61K 47/36

(52) U.S. Cl. CPC A61K 31/7024; A61K 9/06; A61K 47/26; A61K 47/36

(57) ABSTRACT

A jelly-like pharmaceutical composition may include sucralfate hydrate and agar as a jelly base, and optionally further a nonionic dispersant. The agar may be in a range of from 0.1 to 5% by mass. The sucralfate hydrate may be in a range of from 1 to 50% by mass. The nonionic dispersant may be in the composition is from 0.1 to 20% by mass. The nonionic dispersant may be hydroxypropyl starch.

### TECHNICAL FIELD

The present invention relates to a jelly-like pharmaceutical composition containing sucralfate hydrate as an active ingredient.

### BACKGROUND ART

Sucralfate hydrate has been widely used as an anti-peptic ulcer drug in prescription pharmaceuticals or OTC pharmaceuticals.

Sucralfate hydrate is a hydrate of sucrose sulfate ester aluminum salt, composed of a complex of sucrose octasulfate ester and aluminum hydroxide of formula (1), and is white powder poorly soluble in water. Sucralfate hydrate has the effect of promoting the body's own mucosal repair action by selectively binding to ulcerated areas of the gastrointestinal tract to form a protective layer, thereby protecting gastrointestinal mucous membrane from excessive gastric acid or pepsin; neutralizing gastric acid; and inhibiting the activity of enzymes such as pepsin.

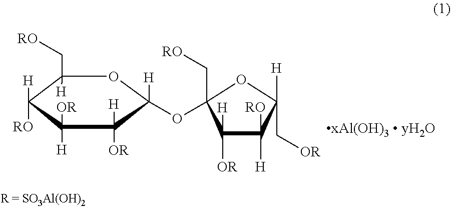

A pharmaceutical containing sucralfate hydrate is produced and sold as a dosage form such as a suspension, granules, or a tablet (for example, Non Patent Literature 1). Since sucralfate hydrate in the suspension is highly dispersed in the stomach, and has high adhesion to the gastric wall, the suspension is the most frequently used in all the sucralfate hydrate preparations, but such preparations have problems in that some sucralfate hydrate remains in the oral cavity to leave the aftertaste of the drug, leaving powdery feeling, and that the contents splash and contaminate when opened. In the case of granules, some of the granules may remain in the oral cavity in the same way as the suspension, and may enter the trachea during ingestion to induce coughing. Tablets may be difficult to take for elderly people or children who have difficulty in swallowing. Water was necessary at the time of ingesting sucralfate hydrate, and the ingestion thereof was difficult in situations and regions where water was not readily available. Accordingly, a preparation which can solve these problems has been desired.

Examples of a medicinal preparation which elderly patients easily take include oral jellies. The Japanese Pharmacopoeia, Eighteenth Edition defines the jellies as non-flowable molded gel-like preparations for oral administration. Generally, a jelly is composed of an active ingredient, a polymer gelling agent, and water, and can contain additives such as a dispersant and a preservative as necessary, depending on the properties of principal agent.

For example, Patent Literature 1 discloses a jelly containing three branched amino acids consisting of isoleucine, leucine, and valine, which can be ingested in a smaller amount and has a good flavor and smooth drinkability, and excellent stability.

Patent Literature 2 discloses that a jelly-like pharmaceutical composition containing a gelling agent, 0.005 to 0.1% by weight parabens, and 0.02 to 0.5% by weight benzoic acid or a salt thereof and having an osmotic pressure of from 1 000 to 3 500 mOsm has high preservative power and is easy to take for good taste and good texture.

Patent Literature 3 discloses a milbemycin oxime-containing jelly-like pharmaceutical composition which contains milbemycin oxime as a medicinally effective ingredient, agar powder as a gelling agent, a sucrose fatty acid ester or a glycerin fatty acid ester as a dispersant, and purified water, and is chemically stable and easily taken.

Although, as a technique related to sucralfate, Patent Literature 4 discloses a gel composition, for example, comprising (A) 18 to 70% by mass sucralfate, (B) 7 to 30% by mass of organic acid, (C) water, and (D) a neutralization thickener, wherein the composition is composed of a dispersant and a dispersoid, the mass ratio of the sucrose octasulfate ester to aluminum in the dispersoid is from 0.3 to 1.4, and pH is from 5.0 to 8.0, it is essential to blend an organic acid; a neutralization thickener such as an alkali metal, and a water-soluble anionic polymer compound, production examples of jellies are not disclosed, and jellies containing sucralfate hydrate are not specifically reported.

### CITATION LIST

### Patent Literature

Patent Literature 1: JP-B-6093181
Patent Literature 2: JP-B-5611672
Patent Literature 3: JP-B-6499970
Patent Literature 4: JP-B-6627485

### Non Patent Literature

Non Patent Literature 1: “Arusarumin (toroku-shohyo) sairyu 90% and Arusarumin naiyoueki 10%” Iyakuhin Intabyufomu, 2019 nen 9 gatsu kaitei (kaitei dai-6 han) (“ULCERLMIN (R) Fine Granule 90% and ULCERLMIN Oral Suspension 10%” Pharmaceutical Interview Form, revised on September 2019 (the revised sixth edition))

### SUMMARY OF INVENTION

### Technical Problem

The present invention relates to a jelly-like pharmaceutical composition containing sucralfate hydrate which can be easily ingested without water and does not cause a remaining feeling of the powder after the ingestion, providing a sucralfate hydrate-containing jelly-like pharmaceutical composition wherein the composition is excellent in stability and moldability, and a method for producing the composition.

### Solution to Problem

The present inventors have earnestly studied a jelly-like pharmaceutical composition containing sucralfate hydrate and found that the adoption of agar as a jelly base enables preparing a good jelly-like pharmaceutical composition, and further blending a nonionic dispersant enables more uniformly dispersing sucralfate hydrate in the preparation to prepare a jelly-like pharmaceutical composition which is excellent in stability and moldability and exhibits good taste and texture. Thus, the present invention has been accomplished.

That is, the present invention relates to the following.
[1] A jelly-like pharmaceutical composition, comprising sucralfate hydrate, wherein the composition comprises agar as a jelly base.
[2] A jelly-like pharmaceutical composition, comprising sucralfate hydrate, wherein the composition comprises agar as a jelly base and a nonionic dispersant.
[3] The jelly-like pharmaceutical composition according to the [1] or [2], wherein a content of agar in the composition is from 0.1 to 5% by mass.
[4] The jelly-like pharmaceutical composition according to the [2] or [3], wherein a content of sucralfate hydrate in the composition is from 1 to 50% by mass, and a content of the nonionic dispersant in the composition is from 0.1 to 20% by mass.
[5] The jelly-like pharmaceutical composition according to the [2] to [4], wherein the nonionic dispersant is hydroxypropyl starch.
[6] The jelly-like pharmaceutical composition according to the [1] to [5], which contains a saccharide.
[7] The jelly-like pharmaceutical composition according to the [6], wherein a content of the saccharide in the composition is from 0.1 to 50% by mass.
[8] The jelly-like pharmaceutical composition according to the [6] or [7], wherein the saccharide is one or more of sorbitol, erythritol, xylitol, and glucose.
[9] A method for producing a jelly-like pharmaceutical composition comprising sucralfate hydrate, comprising a step of mixing a sucralfate suspension into an aqueous solution comprising a jelly base and a nonionic dispersant.

### Advantageous Effects of Invention

According to the present invention, sucralfate hydrate can be highly dispersed, whereby the sedimentation of sucralfate hydrate in the jelly is suppressed, and a jelly-like pharmaceutical composition which is excellent in moldability as the jelly and is easily taken without a bitter taste or a rough (powdery) feeling derived from sucralfate hydrate, can be provided.

### DESCRIPTION OF EMBODIMENTS

Now, the present invention will be described in detail.

The jelly-like pharmaceutical composition of the present invention contains sucralfate hydrate as an active ingredient and agar as a jelly base.

In the present invention, a commercially available sucralfate hydrate is used, however, sucralfate hydrate produced by the production method described in JP-B-44-11673, JP-B-44-16037, JP-B-5-76927, WO 1992/04030, or the like may be used. Sucralfate hydrate is solid, and the particle size thereof is not particularly limited, but fine sucralfate hydrate is preferable since the sucralfate hydrate exhibits drug efficacy by facilitating the binding thereof to an ulcerated areas of the gastrointestinal tract, the sucralfate hydrate does not leave a rough feeling in the oral cavity, and the sucralfate hydrate is prevented from being sedimented in the production process. For example, the sucralfate hydrate having an average particle size of from 0.1 to 150 μm, preferably from 0.1 to 100 μm, more preferably from 0.1 to 50 μm, and further more preferably from 0.1 to 20 μm may be used.

In the present invention, the average particle size means a median size (D₅₀: the particle size corresponding to 50% in a cumulative distribution curve) and is measured based on volume.

The average particle size can be measured by means of a measuring apparatus by a known method, and for example, a method such as laser light scattering particle size distribution measurement or dynamic light scattering particle size distribution measurement.

The amount of sucralfate hydrate blended in the jelly-like pharmaceutical composition of the present invention is not particularly limited, and sucralfate hydrate can be blended preferably at from 1 to 50% by mass, more preferably at from 3 to 30% by mass, further more preferably at from 5 to 25% by mass per unit preparation. The amount to be blended may be one dose, and for example, the amount of sucralfate hydrate to be blended in one preparation may be 1 g.

In the present invention, the jelly-like pharmaceutical composition is a pharmaceutical composition to be a jelly, and is a jelly-like composition containing at least a jelly base and water besides the principal agent. The water content in the composition is usually from 10 to 99% by mass, preferably from 20 to 95% by mass, more preferably from 30 to 90% by mass. The amount of sucralfate hydrate to be blended is expressed in terms of dry matter.

Here, jellies refer to non-flowable preparations for oral administration using polymer gel as a base and having a certain shape (the Japanese Pharmacopoeia, Eighteenth Edition).

In the jelly-like pharmaceutical composition of the present invention, agar is used as the jelly base. For example, if xanthan gum, pectin, gellan gum, carrageenan, carob bean gum, or the like is used as a main jelly base, sufficient moldability is not obtained.

The moldability of the oral jelly in the present invention means: (1) particles of sucralfate hydrate are uniformly dispersed, the entire oral jelly is white and transparent without unevenness, and the overall color is the same and white color which is derived from sucralfate hydrate; (2) the jelly is in the form of gel which is non-flowable and maintains a certain shape; and (3) the syneresis rate is less than or equal to a certain value.

Examples of the agar used in the present invention include polysaccharides consisting of a neutral polysaccharide with β-D-galactopyranose linked at the 1,3-position and 3,6-anhydro-L-galactopyranose linked at the 1,4-position as repeating units (agarose), and an acid polysaccharide containing ionic groups such as sulfate ester groups, pyruvate groups, and methoxyl groups in the backbone skeleton which has the same linking manner as agarose (agaropectin).

The jelly strength of agar is not particularly limited, examples include commercial agar having a jelly strength of from 100 to 1 500 g/cm², and preferable examples include agar having a jelly strength of from 300 to 1 000 g/cm². The jelly strength is measured by a measuring method in accordance with the Japanese Pharmacopoeia. The amount of the agar to be blended in the jelly-like pharmaceutical composition of the present invention is not particularly limited, and the agar can be blended preferably at from 0.1 to 5% by mass, more preferably at from 0.1 to 3% by mass, more preferably at from 0.1 to 2% by mass, and further more preferably at from 0.2 to 1.5% by mass per unit preparation. The amount of the agar to be blended can be optionally increased or decreased to obtain the jelly strength of a desired jelly-like pharmaceutical composition of the present invention.

Instantly soluble agar can be used except the common agars described above. Although, it varies, depending on the type of the agar, instantly soluble agars are dissolved in water at a lower temperature as compared with common agars, preferably at 70° C. or more, and more preferably at 80° C. or more. The jelly strength thereof is from 100 to 800 g/cm². As the jelly strength decreases, the blended amount thereof can be increased. The amount of the agar to be blended in the jelly-like pharmaceutical composition of the present invention is from 0.2 to 5% by mass.

The jelly strength of the jelly-like pharmaceutical composition of the present invention is not particularly limited, but the jelly-like pharmaceutical composition of the present invention is a non-flowable, molded gel-like composition in view of the maintenance of the shape of the jelly and ease of swallowing the jelly. The descriptions of the jelly-like pharmaceutical composition of the present invention is specified in Jellies in the Japanese Pharmacopoeia, the United States Pharmacopeia, or the European pharmacopoeia. In order to obtain desired jelly strength in the jelly-like pharmaceutical composition of the present invention, the amount of the agar to be blended has to be changed. For example, if the blended amount is large, the strength can be increased to make the composition pudding-like, gummi-like, or adzuki bean jelly-like. If the blended amount is small, the strength can be reduced to make the composition gel-like.

It is preferable to blend a nonionic dispersant as the dispersant in the jelly-like pharmaceutical composition of the present invention. Although sucralfate hydrate is likely to sediment in an aqueous solution, resulting in the separation, the addition of the nonionic dispersant increases the viscosity of the solution and suppresses the sedimentation of sucralfate hydrate to disperse sucralfate hydrate.

Examples of the nonionic dispersant include celluloses such as methylcellulose, ethylcellulose, hydroxyethylcellulose, hydroxypropylcellulose, and hydroxypropylmethylcellulose; and nonionic modified starches such as acetylated adipate crosslinked starch, acetylated phosphate crosslinked starch, acetylated oxidized starch, hydroxypropyl starch, oxidized starch, starch acetate, phosphorylated starch, and hydroxypropylated phosphate crosslinked starch. Preferable examples include nonionic modified starches, and more preferable examples include hydroxypropyl starch from the viewpoint of the effect of preventing the sedimentation of sucralfate.

The amount of the nonionic dispersant to be blended in the jelly-like pharmaceutical composition of the present invention is not particularly limited, and can be preferably from 0.1 to 20% by mass, more preferably from 0.2 to 15% by mass, and further more preferably from 0.3 to 10% by mass per unit preparation.

A saccharide can be further blended into the jelly-like pharmaceutical composition of the present invention. This enables leading to good jelly strength, a good syneresis rate, good taste, and good texture.

Examples of the saccharide include sugars such as sucrose, palatinose, and fructo-oligosaccharide; starch saccharides such as glucose, maltose, starch syrup, and fructose; lactoses such as lactose, isomerized lactose, and reduced lactose; and sugar alcohols such as sorbitol, mannitol, reduced maltose starch syrup (maltitol), saccharified reduced starch, xylitol, reduced palatinose, erythritol, and sorbitol. Starch saccharides and sugar alcohols are preferable, more preferable examples include erythritol, xylitol, sorbitol, and glucose, further more preferable examples include erythritol and sorbitol, and further more preferable examples include sorbitol from the viewpoint of taste and texture. These saccharides may be used alone or in combination of two or more.

The amount of the saccharide to be blended in the jelly-like pharmaceutical composition of the present invention is not particularly limited, and is preferably from 0.1 to 50% by mass, more preferably from 0.5 to 40% by mass, further preferably from 1 to 30% by mass, and the most preferably from 3 to 20% by mass per unit preparation.

Pharmaceutical additive ingredients can be appropriately used for the jelly-like pharmaceutical composition of the present invention as needed. For example, a preservative, a sweetener, a flavor, an organic solvent, a pH adjustor, and the like can be blended.

Examples of the preservative include one or more selected from the group consisting of benzoic acids such as benzoic acid, benzyl benzoate, and sodium benzoate; parabens such as methylparaben, ethylparaben, propylparaben, isopropylparaben, butylparaben, isobutylparaben and benzylparaben; and benzalkonium chloride, cetylpyridinium chloride, benzethonium chloride, aminoethylsulfonic acid. Preferable examples include parabens, and more preferable examples include one or more selected from the group consisting of methylparaben, ethylparaben, propylparaben, isopropylparaben, butylparaben, and isobutylparaben.

As long as the amount of the preservative to be blended in the jelly-like pharmaceutical composition of the present invention is within the usage regulations, the amount is not particularly limited, and the amount can be preferably from 0.00001 to 2% by mass, more preferably from 0.0001 to 1.5% by mass, and further more preferably from 0.002 to 0.5% by mass per unit preparation.

As the sweetener, one or more sweeteners selected from the group consisting of sugar, oligosaccharide, maltitol, erythritol, sorbitol, xylitol, aspartame, acesulfame potassium, sucralose, stevia, and the like can be used.

The amount of the sweetener to be blended in the jelly-like pharmaceutical composition of the present invention can be from 0.001 to 40% by mass per unit preparation.

As the flavor, for example, flavors having flavors such as an apple, an orange, a grapefruit, a strawberry, a peach, a lemon, and yogurt can be used.

The amount of the flavor to be blended in the jelly-like pharmaceutical composition of the present invention can be 0.0001 to 2% by mass per unit preparation.

The organic solvent is used for dissolving lipophilic ingredients such as the preservative and the flavor. For examples, an alcohol or a polyhydric alcohol can be used, examples of the alcohol include ethanol, and examples of the polyhydric alcohol include glycerin, ethylene glycol, and propylene glycol. Preferable examples include ethanol, glycerin, and mixed liquid thereof.

A pH adjustor which can adjust the pH to 4 or more is preferred, and sodium hydroxide, potassium hydroxide, sodium hydroxide, calcium hydroxide, magnesium hydroxide, sodium carbonate, sodium hydrogen carbonate, ammonia water, sodium citrate, sodium hydrogen citrate, trisodium phosphate, sodium monohydrogen phosphate, sodium dihydrogen phosphate, tripotassium phosphate, potassium monohydrogen phosphate, potassium dihydrogen phosphate, monoethanolamine, or the like may be mentioned. Since the blended amount is small, sodium hydroxide does not have the effect of crosslinking an acidic polymer, and the pH adjustor is preferably sodium hydroxide.

A pharmaceutical additive other than the above can be blended in the jelly-like pharmaceutical composition of the present invention to maintain desired physical properties. For example, a gelling agent can be added for preventing the jelly strength from decreasing and improving the jelly strength. The gelling agent may, for example, be xanthan gum, carrageenan, locust bean gum, pectin, gellan gum, and the blended amount is from 0.001 to 1%.

For example, the jelly-like pharmaceutical composition of the present invention can be sealed in a cup container, a CHEER PACK®, a stick type packaging container so as to be divided into doses and provided in a form which patients take easily.

The jelly-like pharmaceutical composition of the present invention is excellent in moldability. Sucralfate hydrate is dispersed at a uniform concentration in a jelly filled in a container, and the jelly is overall white without shading as a whole.

The jelly-like pharmaceutical composition of the present invention is characterized in that water is scarcely separated therefrom. The syneresis rate indicates the separation of water. The syneresis rate of the jelly-like pharmaceutical composition of the present invention is from 0 to 15%, preferably from 0.1 to 10%. If the jelly-like pharmaceutical composition is filled into a stick-shaped packet, syneresis water does not flow out or attach to the body and clothes at the time of ingestion.

When the holding time at about 55° C. or more is extended in the production process described below, the decomposition of the agar progresses to reduce the strength of the jelly-like pharmaceutical composition of the present invention, and thus the pH of the present composition is preferably higher, and is from 3 to 8, preferably from 4 to 7, and more preferably from 5 to 7.

The jelly-like pharmaceutical composition of the present invention has a pharmaceutical effect which is equivalent to the existing sucralfate suspension. Since the acid-neutralizing capacities or the enzyme adsorption capacities are equivalent, the equivalent effect can be confirmed. The acid-neutralizing capacity is 130 mL or more, preferably 150 mL or more, per 1 g of sucralfate (one dose) in the test of the Japanese Pharmacopoeia.

The jelly-like pharmaceutical composition of the present invention can be produced by a known method for producing a jelly. For example, agar is dissolved in water at about 85° C. or more, and a nonionic dispersant, a sweetener, and sucralfate hydrate are added thereto to prepare a base solution. If hydroxypropyl starch is used as the dispersant, the dispersant is added to water, and the aqueous solution is then gelatinized at about 75° C. or more to adjust the viscosity of the solution. The temperature of the solution to which both hydroxypropyl starch and agar are added may be adjusted to 85° C. or more. If instantly soluble agar is used, the instantly soluble agar is however dissolved at 70° C. or more.

When sucralfate hydrate is added, it is preferable to prepare a suspension of sucralfate hydrate beforehand and add the suspension. The suspension of sucralfate hydrate only needs to have a flowable viscosity, and the concentration of sucralfate hydrate is from 10 to 40%. It is preferred to warm the suspension of sucralfate hydrate to 30 to 70° C. and add the suspension.

Further, if lipophilic ingredients such as the preservative and the flavor are added, they can be dissolved in the organic solvent separately and added to the above-described base solution, followed by uniformly mixing the solution to prepare a jelly solution. When pH of the pharmaceutical composition of the present invention is adjusted, the pH adjustor can be added as it is or as an aqueous solution in the preparation of the liquid for suspending sucralfate hydrate or the jelly solution.

The jelly solution can be subsequently filled into a container at 50 to 80° C. and then cooled to room temperature to produce the jelly-like pharmaceutical composition of the present invention. If a disinfection step is carried out, the jelly solution can be heat-treated in a step after the preparation of the jelly solution and before the filling of the jelly solution, or the solution can be sterilized after the container is filled with the solution.

### EXAMPLES

Hereinafter, the present invention will be described in more detail with Examples, Comparative Example, and Test Examples, but the present invention is by no means limited thereto.

1. Effects of Preventing Sedimentation of Sucralfate Hydrate with Various Dispersants

Each dispersant shown in Table 1 was added to purified water in a predetermined amount and dissolved therein to prepare a base solution. Sucralfate hydrate was added to this solution and dispersed therein with a homomixer to prepare a drug solution.

The effect of preventing the sedimentation of sucralfate hydrate with the dispersant in each drug solution was evaluated. The proportions of the respective ingredients are that the amount of sucralfate hydrate which is equivalent amount of 10% sucralfate in terms of dry matter, the amount of the dispersant shown in Table 1 and the amount of water to be the total amount of 100% (here & used in the table means % by mass).

The drug solution was left to stand for 1 day, followed by visual evaluation. Table 1 summarizes the results.

TABLE 1

| | Added | | | Added | |
|---|---|---|---|---|---|
| | amount of | | | amount of | |
| Drug | sucralfate | | Added | purified | |
| solution | hydrate | Dispersant | amount | water | Result |
| 1 | 11.11% | Hydroxypropyl | 2.50% | 86.39% | The sedimentation |
| | | starch | | | was suppressed. |
| 2 | 11.11% | Sodium | 1.00% | 87.89% | Artificial salmon roe- |
| | | polyacrylate | | | like (*1) |

(*1): An artificial salmon roe is a roe-like article having a film containing an aqueous solution of sodium alginate as the main ingredient on the surface and containing carrageenan and the like as the contents.

When hydroxypropyl starch, which was a nonionic dispersant, was used as the dispersant, the highest effect of preventing the sedimentation of sucralfate hydrate was exhibited. It was found that if the ionic dispersant was added, the drug solution became artificial salmon roe-like, and was problematically prepared into a jelly.

### 2. Preparation of Jelly-Like Pharmaceutical Composition

### Example 1

Hydroxypropyl starch, agar, erythritol, acesulfame potassium, and sucralose were added to purified water, and the temperature was raised to about 90° C. to dissolve all the ingredients, and a base solution was thereby prepared. Sucralfate hydrate was added to water and dispersed by a homomixer to prepare a drug solution. Methylparaben and a flavor were dissolved in ethanol, and the mixture was added to glycerin to prepare an oily solution. The drug solution was added to the base solution, followed by mixing the solution, and the oily solution was further added thereto, followed by mixing the solution, to prepare a jelly solution. The jelly solution was cooled to room temperature to prepare a jelly-like pharmaceutical composition. The pH was 4.1. Table 2 shows the amounts of the ingredients blended (% by mass). As the sucralfate hydrate, mild sucralfate (about 6 μm in average particle size), which was available from Fuji Chemical Industries Co., Ltd., was used. As the agar, agar which had a jelly strength of about 600 g/cm², and was available from Ina Food Industry Co., Ltd., was used.

TABLE 2

| Ingredient | Blended amount (% by mass) |
|---|---|
| Sucralfate hydrate | 11 |
| Agar | 0.6 |
| Erythritol | 5 |
| Hydroxypropyl starch | 3 |
| Water | 74.671 |
| Acesulfame potassium | 0.007 |
| Sucralose | 0.003 |
| Methylparaben | 0.05 |
| Lemon flavoring | 0.02 |
| Ethanol | 0.649 |
| Glycerin | 5 |

### Examples 2 to 4

Jelly-like pharmaceutical compositions were produced in the same manner as in Example 1 except that erythritol among the ingredients described Example 1 was changed into the saccharide described in Table 3.

### Comparative Examples 1 to 5

Jelly-like pharmaceutical compositions were produced in the same manner as in Example 1 except that agar among the ingredients described Example 1 was changed into the jelly bases described in Table 3.

### [Evaluation of Moldability, Taste, and Texture]

The moldabilities and the tastes and textures of the jelly-like pharmaceutical compositions of Examples 1 to 4 and Comparative Examples 1 to 5 were evaluated. The moldabilities were visually evaluated. The evaluation of the tastes and textures were evaluated by sensory tests. Table 3 shows the results.

TABLE 3

| | | | | | Comparative |
|---|---|---|---|---|---|
| | Example 1 | Example 2 | Example 3 | Example 4 | Example 1 |
| Jelly base | Agar | Agar | Agar | Agar | Xanthan gum |
| Dispersant | Hydroxypropyl | Hydroxypropyl | Hydroxypropyl | Hydroxypropyl | Hydroxypropyl |
| | starch | starch | starch | starch | starch |
| Saccharide | Erythritol | Sugar | Maltitol | Sorbitol | Erythritol |
| Moldability | Good (*1) | Good | Good | Good | Artificial |
| | | | | | salmon roe- |
| Taste and | Good | Good | Good | Good | like (*3) |
| texture | | | | | —(*2) |

| | Comparative | Comparative | Comparative | Comparative |
|---|---|---|---|---|
| | Example 2 | Example 3 | Example 4 | Example 5 |
| Jelly base | Pectin | Gellan gum | Carrageenan | Carob bean gum |
| Dispersant | Hydroxypropyl | Hydroxypropyl | Hydroxypropyl | Hydroxypropyl |
| | starch | starch | starch | starch |
| Saccharide | Erythritol | Erythritol | Erythritol | Erythritol |
| Moldability | Artificial | Artificial | Artificial | Gel |
| | salmon roe- | salmon roe- | salmon roe- | separation |
| Taste and | like | like | like | |
| texture | — | — | — | — |

(*1): Uniformly jelly-like without separation
(*2)Not measured
(*3): An artificial salmon roe is a roe-like article having a film containing an aqueous solution of sodium alginate as the main ingredient on the surface and containing carrageenan and the like as the contents.

The preparations of Examples 1 to 4 exhibited good moldabilities as jelly-like pharmaceutical compositions. The jelly-like pharmaceutical composition of Example 1 exhibited good taste and texture.

It was not possible to prepare jelly-like pharmaceutical composition in any of the preparations of Comparative Examples 1 to 5 which use the jelly bases other than agar.

### Examples 5 to 6

Jelly-like pharmaceutical compositions were produced in the same manner as in Example 1 except that the amounts of agar and water among the ingredients described in Example 1 were adjusted to change the amount of agar blended into the amounts described in Table 4.

TABLE 4

| | Example 5 | Example 6 |
|---|---|---|
| Jelly base | Agar | Agar |
| Blended amount | 2% | 3% |
| Moldability | Good | Good |
| Taste and texture | Good | Good |

The preparations of Examples 5 to 6 exhibited good moldabilities and good tastes and textures as jelly-like pharmaceutical compositions.

### Example 7

Hydroxypropyl starch, agar, sorbitol, and acesulfame potassium were added to purified water, and the temperature was raised to about 85° C. to dissolve all the ingredients, and a base solution was thereby prepared. Sucralfate hydrate and sodium hydroxide were added to water and dispersed with a homomixer to prepare a drug solution. Ethylparaben, propylparaben, and a flavor were dissolved in ethanol, and the mixture was added to glycerin to prepare an oily solution. The drug solution was added to the base solution, followed by mixing the solution, and the oily solution was further added thereto, followed by mixing the solution, to prepare a jelly solution. The jelly solution was filled into a stick-shaped three-side seal bag while the jelly solution was maintained at 40° C. or more. The jelly solution was cooled to room temperature to prepare a jelly-like pharmaceutical composition. Table 4 shows the blending amounts of the ingredients (% by mass). As the sucralfate hydrate, mild sucralfate (about 6 μm in average particle size), which was available from Fuji Chemical Industries Co., Ltd., was used. As the agar, agar which had a jelly strength of about 830 g/cm², and was available from Ina Food Industry Co., Ltd. was used.

TABLE 5

| Ingredient | Blended amount (% by mass) |
|---|---|
| Sucralfate hydrate | 11.148*¹ |
| Sodium hydroxide | 0.45 |
| Agar | 0.4 |
| Sorbitol | 10 |
| Hydroxypropyl starch | 2 |
| Water | Suitable amount*² |
| Acesulfame potassium | 0.002 |
| Ethylparaben | 0.01 |
| Propylparaben | 0.005 |
| Lemon flavoring | 0.02 |
| Ethanol | 0.649 |
| Glycerin | 5 |

*¹Equivalent to 10% by mass in terms of dry matter
*²Added so that the total amount was adjusted to 100%

### (Stability)

The compositions were maintained under the conditions of a temperature of 40° C. and a humidity of 75 for 1 month to obtain the analytical results shown in Table 6.

TABLE 6

| | Example 7 | |
|---|---|---|
| | Initial | 1 month |
| Description | White jelly which tastes sweet | Not |
| | and smells of lemon. | changed |
| Texture | Feeling the shape in the mouth, | Not |
| | having hardness enough to be | changed |
| | crushed with the tongue | |
| pH | 6.23 | 6.18 |
| Disintegrability | 30 minutes | 30 minutes |
| | or more | or more |
| Syneresis rate | 4.8% | 6.9% |
| Acid-neutralizing | 172 | 176 |
| capacity | | |
| Related Substance | 0.044 | 0.044 |

The jelly-like pharmaceutical composition of Example 7 was stable without changing from the “Initial” even under an acceleration condition for 1 month.

1. A jelly-like pharmaceutical composition, comprising:
sucralfate hydrate; and
agar as a jelly base.

2. A jelly-like pharmaceutical composition, comprising:
sucralfate hydrate;
agar as a jelly base; and
a nonionic dispersant.

3. The composition of claim 1, comprising the agar in a range of from 0.1 to 5% by mass.

4. The composition of claim 1, comprising the sucralfate hydrate in a range of from 1 to 50% by mass.

5. The composition of claim 2, wherein the nonionic dispersant is hydroxypropyl starch.

6. The composition of claim 1, further comprising:
a saccharide.

7. The composition according of claim 6, comprising the saccharide in a range of from 0.1 to 50% by mass.

8. The composition of claim 6, wherein the saccharide is sorbitol, erythritol, xylitol, and/or glucose.

9. A method for producing a jelly-like pharmaceutical composition, the method comprising:
mixing a sucralfate suspension into an aqueous solution comprising a jelly base and a nonionic dispersant,
wherein the jelly-like pharmaceutical composition comprises sucralfate hydrate.

10. The composition of claim 2, comprising the agar in a range of from 0.1 to 5% by mass.

11. The composition of claim 2, comprising the sucralfate hydrate in a range of from 1 to 50% by mass.

12. The composition of claim 2, comprising the nonionic dispersant in a range of from 0.1 to 20% by mass.

13. The composition of claim 12, wherein the nonionic dispersant is hydroxypropyl starch.

14. The composition of claim 2, further comprising:
a saccharide.

15. The composition of claim 14, wherein the saccharide is sorbitol, erythritol, xylitol, and/or glucose.

16. The composition of claim 2, comprising:
the sucralfate hydrate in a range of from 1 to 50% by mass; and
the nonionic dispersant in a range of from 0.1 to 20% by mass.

17. The method of claim 10, wherein the jelly-like pharmaceutical composition comprises, by mass based on total jelly-like pharmaceutical composition mass:
the agar in a range of from 0.1 to 5%; and
the sucralfate hydrate in a range of from 1 to 50%.

18. The method of claim 10, wherein the aqueous solution further comprises a saccharide.

19. The method of claim 18, wherein the saccharide is present in the aqueous solution in a range of from 1 to 50% by mass, based on total aqueous solution mass.
